(19) United States
(12) Patent Application Publication
(10) Pub. No.: US 2026/0051381 A1
(43) Pub. Date: Feb. 19, 2026

(54) AUTOMATED POPULATION DEVELOPMENT FOR SEARCH QUERIES

(71) Applicant: Express Scripts Strategic Development, Inc., St. Louis, MO (US)

(72) Inventors: Johanna Bellon, Stamford, CT (US); Sarah Azhar, Great Falls, VA (US); Subhasree Devendran, Tampa, FL (US); Bhavya Shah, West Orange, NJ (US); Hari C. Narayanan, Livingston, NJ (US); Bijuna S. Pramila, Livingston, NJ (US)

(21) Appl. No.: 18/808,189

(22) Filed: Aug. 19, 2024

(51) Int. Cl. G16H 20/10; G06F 16/28

(52) U.S. Cl. CPC G16H 20/10; G06F 16/285

(57) ABSTRACT

A method includes, in response to detecting a set of user inputs, generating a first set of rules, transforming a graphical user interface, and transmitting the first set of rules. The method includes, in response to a determination that the first set of rules have been authorized, storing the first set of rules. The method includes loading a first set of user records and generating a second set of user records using the first set of rules. The method includes determining a classification for a respective record using the first set of rules. The method includes, in response to a determination that the classification of the respective record is a first class, generating a first set of goals, associating the first set of goals with the respective record, generating a first set of actions, and associating the first set of actions with the respective record.

### FIELD

The present disclosure relates to database query creation and more particularly to iterative formation of a database query and processing of query results.

### BACKGROUND

In a healthcare context, healthcare providers frequently need to create individualized plans for healthcare patients to address patient needs. These plans can be helpful for a number of patient needs: for example, in preparation for a surgery, surgery recovery, or long-term injury maintenance.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

A computerized method for classifying records across locations includes generating an administrator graphical user interface for presentation to an administrative user at a first user device. The method includes, in response to detecting, via the administrator graphical user interface, a set of user inputs from the administrative user, generating a first set of rules, transforming the administrator graphical user interface to display a set of user interface elements corresponding to the first set of rules, transmitting the first set of rules to a first data storage location, and in response to a determination that the first set of rules have been authorized, storing the first set of rules in the first data storage location. The method includes loading a first set of user records from a set of data storage locations. The method includes generating a second set of user records from the first set of user records using the first set of rules. The method includes determining a classification for a respective record of the second set of user records using the first set of rules. The respective record includes data associated with a second user. The method includes loading a second set of rules from the first data storage location. The method includes, in response to a determination that the classification of the respective record is a first class, generating a first set of goals based on the second set of rules and the first class and associating the first set of goals with the respective record. The method includes, in response to a determination that the classification of the respective record is a first class, generating a first set of actions based on the second set of rules and the first class and associating the first set of actions with the respective record. The method includes, in response to a determination that the classification of the respective record is a first class, transmitting the first set of goals and the first set of actions for access at a second user device.

In other features, the method includes, in response to a determination that the classification of the respective record is a second class, generating a second set of goals based on the second set of rules and the second class, associating the second set of goals with the respective record, and generating a second set of actions based on the second set of rules and the second class and associating the second set of actions with the respective record.

In other features, the method includes transmitting the second set of goals and the second set of actions for access at a second user device. In other features, the method includes transforming, at the second user device, a second user interface to display the first set of goals and the first set of actions, or the second set of goals and the second set of actions. In other features, the method includes transforming the second user interface to display a first set of user prompts. In other features, the method includes, in response to detecting, at the second user device, a second set of user inputs from the second user corresponding to the first set of user prompts, generating a third set of goals based on the second set of rules and associating the third set of goals with the respective record, and generating a third set of actions based on the second set of rules and associating the third set of actions with the respective record.

In other features, the method includes in response to the second set of user inputs, changing the classification of the respective record to a third class. The third class is different from the first class and the second class. In other features, the second set of user inputs corresponds to a set of answers to the first set of user prompts. In other features, the method includes displaying the first set of user prompts in response to displaying a user account associated with the respective record.

In other features, the method includes in response to the determination that the classification of the respective record is the first class, including a first set of questions in the first set of user prompts. In other features, the method includes in response to the determination that the classification of the respective record is the second class, including a second set of questions in the first set of user prompts. At least one question of the second set of questions differs from at least one question of the first set of questions.

In other features, the first data storage location stores data in a non-relational database. In other features, saving data to the first data storage location is performed by a first function. Loading data from the first data storage location is performed by a second function. In other features, the set of data storage locations is remote from the first data storage location and includes a plurality of storage locations.

In other features, the method includes in response to a determination that the respective record has met one or more criteria of a set of criteria, generating a fourth set of goals based on the second set of rules and associating the fourth set of goals with the respective record. In other features, the method includes in response to a determination that the respective record has met one or more criteria of a set of criteria, generating a fourth set of actions based on the second set of rules and associating the fourth set of actions with the respective record. The fourth set of goals and the fourth set of actions are based on a current classification of the respective record.

In other features, the set of criteria includes a criterion that is met when the respective record includes an indication of a first health concern. In other features, the set of criteria includes a criterion that is met when the respective record includes an indication of a second health concern. In other features, the set of criteria includes a criterion that is met when the respective record includes an indication of a third health concern.

A computer system comprises memory hardware configured to store instructions and processor hardware configured to execute the instructions stored in the memory hardware. The instructions include generating an administrator graphical user interface for presentation to an administrative user at a first user device. The instructions include, in response to detecting, via the administrator graphical user interface, a set of user inputs from the administrative user, generating a first set of rules, transforming the administrator graphical user interface to display a set of user interface elements corresponding to the first set of rules, transmitting the first set of rules to a first data storage location, and in response to a determination that the first set of rules have been authorized, storing the first set of rules in the first data storage location. The instructions include loading a first set of user records from a set of data storage locations. The instructions include generating a second set of user records from the first set of user records using the first set of rules. The instructions include determining a classification for a respective record of the second set of user records using the first set of rules. The respective record includes data associated with a second user. The instructions include loading a second set of rules from the first data storage location. The instructions include, in response to a determination that the classification of the respective record is a first class, generating a first set of goals based on the second set of rules and the first class and associating the first set of goals with the respective record. The instructions include, in response to a determination that the classification of the respective record is a first class, generating a first set of actions based on the second set of rules and the first class and associating the first set of actions with the respective record. The instructions include, in response to a determination that the classification of the respective record is a first class, transmitting the first set of goals and the first set of actions for access at a second user device.

In other features, the instructions include, in response to a determination that the classification of the respective record is a second class, generating a second set of goals based on the second set of rules and the second class, associating the second set of goals with the respective record, generating a second set of actions based on the second set of rules and the second class, and associating the second set of actions with the respective record.

In other features, the instructions include, transmitting the second set of goals and the second set of actions for access at a second user device. In other features, the instructions include transforming, at the second user device, a second user interface to display the first set of goals and the first set of actions, or the second set of goals and the second set of actions. In other features, the instructions include transforming the second user interface to display a first set of user prompts. In other features, the instructions include, in response to detecting, at the second user device, a second set of user inputs from the second user corresponding to the first set of user prompts, generating a third set of goals based on the second set of rules, associating the third set of goals with the respective record, generating a third set of actions based on the second set of rules, and associating the third set of actions with the respective record.

In other features, the instructions include, in response to the second set of user inputs, changing the classification of the respective record to a third class. The third class is different from the first class and the second class. The second set of user inputs corresponds to a set of answers to the first set of user prompts.

A non-transitory computer-readable storage medium stores processor-executable instructions. The instructions include generating an administrator graphical user interface for presentation to an administrative user at a first user device. The instructions include, in response to detecting, via the administrator graphical user interface, a set of user inputs from the administrative user, generating a first set of rules, transforming the administrator graphical user interface to display a set of user interface elements corresponding to the first set of rules, transmitting the first set of rules to a first data storage location, and in response to a determination that the first set of rules have been authorized, storing the first set of rules in the first data storage location. The instructions include loading a first set of user records from a set of data storage locations. The instructions include generating a second set of user records from the first set of user records using the first set of rules. The instructions include determining a classification for a respective record of the second set of user records using the first set of rules. The respective record includes data associated with a second user. The instructions include loading a second set of rules from the first data storage location. The instructions include, in response to a determination that the classification of the respective record is a first class, generating a first set of goals based on the second set of rules and the first class and associating the first set of goals with the respective record. The instructions include, in response to a determination that the classification of the respective record is a first class, generating a first set of actions based on the second set of rules and the first class and associating the first set of actions with the respective record. The instructions include, in response to a determination that the classification of the respective record is a first class, transmitting the first set of goals and the first set of actions for access at a second user device.

In other features, the instructions stored in the non-transitory computer-readable storage medium include, in response to a determination that the classification of the respective record is a second class, generating a second set of goals based on the second set of rules and the second class, associating the second set of goals with the respective record, generating a second set of actions based on the second set of rules and the second class, and associating the second set of actions with the respective record.

In other features, the instructions stored in the non-transitory computer-readable storage medium include, transmitting the second set of goals and the second set of actions for access at a second user device. In other features, the instructions include transforming, at the second user device, a second user interface to display the first set of goals and the first set of actions, or the second set of goals and the second set of actions. In other features, the instructions include transforming the second user interface to display a first set of user prompts. In other features, the instructions include, in response to detecting, at the second user device, a second set of user inputs from the second user corresponding to the first set of user prompts, generating a third set of goals based on the second set of rules, associating the third set of goals with the respective record, generating a third set of actions based on the second set of rules, and associating the third set of actions with the respective record.

In other features, the instructions stored in the non-transitory computer-readable storage medium include, in response to the second set of user inputs, changing the classification of the respective record to a third class. The third class is different from the first class and the second class. The second set of user inputs corresponds to a set of answers to the first set of user prompts.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 is a functional block diagram of an example system including a high-volume pharmacy.

FIG. 2 is a functional block diagram of an example pharmacy fulfillment device, which may be deployed within the system of FIG. 1.

FIG. 3 is a functional block diagram of an example order processing device, which may be deployed within the system of FIG. 1.

FIG. 4A is a functional block diagram of an example system for developing a population and generating action hierarchies.

FIG. 4B is a functional block diagram of an example system for developing a population and generating action hierarchies.

FIG. 5A is a flowchart of an example population development and report generation method.

FIG. 5B is a flowchart of an example population development and report generation method.

FIGS. 6A-6B together are a flowchart of an example population development and action hierarchy generation method.

FIG. 7 is a flowchart of an example display transformation method.

FIG. 8 is a functional block diagram of example machine learning model training and usage.

FIG. 9 is a functional block diagram of an example neural network.

FIG. 10 is a functional block diagram of an example neural network.

FIG. 11 is a functional block diagram of an example long short-term memory neural network.

In the drawings, reference numbers may be reused to identify similar and/or identical elements.

### DETAILED DESCRIPTION

### Introduction

The present disclosure describes generating action hierarchies for a generated population of user records. In some implementations, an analyst user device implements (via electronic circuitry processing or other machine processing) a set of rules to process groups of user records and associated user data. The creation and modification of rules and predictive data models often requires technical expertise (such as extensive coding knowledge) as well as significant time and resources. Generating rules and modifications for data models via a graphical user interface reduces the complexity of implementing the necessary rules. In some implementations, the set of rules is created via an analyst graphical user interface and/or retrieved from a rules database.

One or more predictive models may be used to analyze data records and generate a subset of user records based on user records to which a set of action hierarchies are applicable. In some implementations, the set of rules can be used to narrow a group of user records to those associated with a specified condition (for example, knee or back pain) and classify patients (e.g., patient electronic records) into subcategories based on the intensity of the condition. Based on the implemented set of rules and subcategories combined with the predictive model processing, each user record can be associated an individualized action hierarchy. In some implementations, the action hierarchy can be updated as needed by an administrator user or in response to additional data associated with a particular user record. In some implementations, action hierarchies are created in real-time in response to user input (for example, a user adds a new record and generates an action hierarchy). In some implementations, action hierarchies are created in batches (for example, in response to an automatic process that looks for new data records that have not yet been analyzed).

For additional context, the entire disclosures of U.S. application Ser. No. 18/101,935 filed 26 Jan. 2023 (Attorney Docket No. ESRX-363US1) and U.S. application Ser. No. 18/205,203 filed 2 Jun. 2023 (Attorney Docket No. ESRX-370US1) are hereby incorporated by reference.

### High-Volume Pharmacy

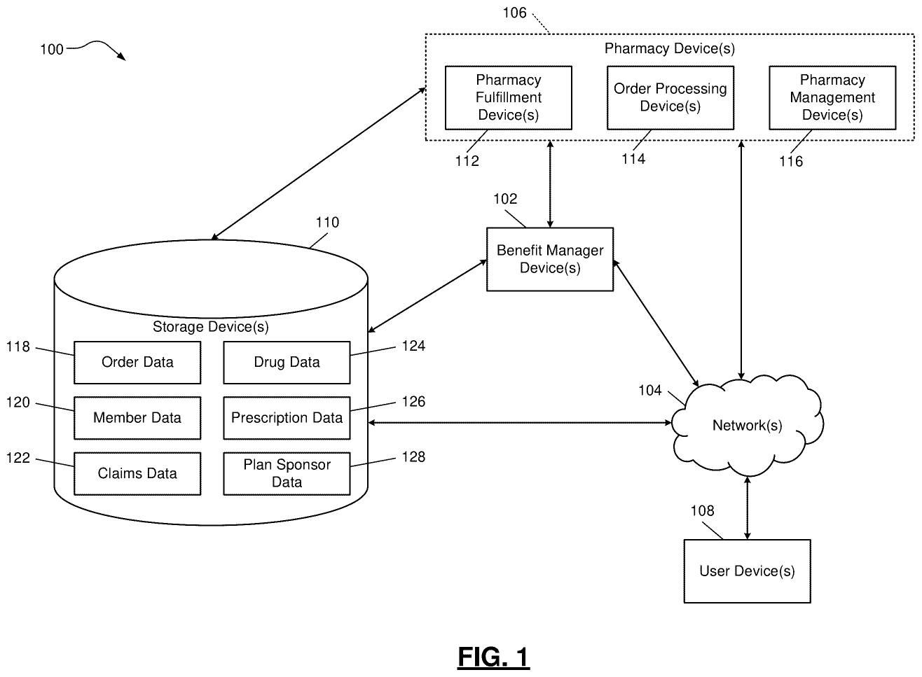

FIG. 1 is a block diagram of an example implementation of a system 100 for a high-volume pharmacy. While the system 100 is generally described as being deployed in a high-volume pharmacy or a fulfillment center (for example, a mail order pharmacy, a direct delivery pharmacy, etc.), the system 100 and/or components of the system 100 may otherwise be deployed (for example, in a lower-volume pharmacy, etc.). A high-volume pharmacy may be a pharmacy that is capable of filling at least some prescriptions mechanically. The system 100 may include a benefit manager device 102 and a pharmacy device 106 in communication with each other directly and/or over a network 104.

The system 100 may also include one or more user device(s) 108. A user, such as a pharmacist, patient, data analyst, health plan administrator, etc., may access the benefit manager device 102 or the pharmacy device 106 using the user device 108. The user device 108 may be a desktop computer, a laptop computer, a tablet, a smartphone, etc.

The benefit manager device 102 is a device operated by an entity that is at least partially responsible for creation and/or management of the pharmacy or drug benefit. While the entity operating the benefit manager device 102 is typically a pharmacy benefit manager (PBM), other entities may operate the benefit manager device 102 on behalf of themselves or other entities (such as PBMs). For example, the benefit manager device 102 may be operated by a health plan, a retail pharmacy chain, a drug wholesaler, a data analytics or other type of software-related company, etc. In some implementations, a PBM that provides the pharmacy benefit may provide one or more additional benefits including a medical or health benefit, a dental benefit, a vision benefit, a wellness benefit, a radiology benefit, a pet care benefit, an insurance benefit, a long-term care benefit, a nursing home benefit, etc. The PBM may, in addition to its PBM operations, operate one or more pharmacies. The pharmacies may be retail pharmacies, mail order pharmacies, etc.

Some of the operations of the PBM that operates the benefit manager device 102 may include the following activities and processes. A member (or a person on behalf of the member) of a pharmacy benefit plan may obtain a prescription drug at a retail pharmacy location (e.g., a location of a physical store) from a pharmacist or a pharmacist technician. The member may also obtain the prescription drug through mail order drug delivery from a mail order pharmacy location, such as the system 100. In some implementations, the member may obtain the prescription drug directly or indirectly through the use of a machine, such as a kiosk, a vending unit, a mobile electronic device, or a different type of mechanical device, electrical device, electronic communication device, and/or computing device. Such a machine may be filled with the prescription drug in prescription packaging, which may include multiple prescription components, by the system 100. The pharmacy benefit plan is administered by or through the benefit manager device 102.

The member may have a copayment for the prescription drug that reflects an amount of money that the member is responsible to pay the pharmacy for the prescription drug. The money paid by the member to the pharmacy may come from, as examples, personal funds of the member, a health savings account (HSA) of the member or the member's family, a health reimbursement arrangement (HRA) of the member or the member's family, or a flexible spending account (FSA) of the member or the member's family. In some instances, an employer of the member may directly or indirectly fund or reimburse the member for the copayments.

The amount of the copayment required by the member may vary across different pharmacy benefit plans having different plan sponsors or clients and/or for different prescription drugs. The member's copayment may be a flat copayment (in one example, $10), coinsurance (in one example, 10%), and/or a deductible (for example, responsibility for the first $500 of annual prescription drug expense, etc.) for certain prescription drugs, certain types and/or classes of prescription drugs, and/or all prescription drugs. The copayment may be stored in a storage device 110 or determined by the benefit manager device 102.

In some instances, the member may not pay the copayment or may only pay a portion of the copayment for the prescription drug. For example, if a usual and customary cost for a generic version of a prescription drug is $4, and the member's flat copayment is $20 for the prescription drug, the member may only need to pay $4 to receive the prescription drug. In another example involving a worker's compensation claim, no copayment may be due by the member for the prescription drug.

In addition, copayments may also vary based on different delivery channels for the prescription drug. For example, the copayment for receiving the prescription drug from a mail order pharmacy location may be less than the copayment for receiving the prescription drug from a retail pharmacy location.

In conjunction with receiving a copayment (if any) from the member and dispensing the prescription drug to the member, the pharmacy submits a claim to the PBM for the prescription drug. After receiving the claim, the PBM (such as by using the benefit manager device 102) may perform certain adjudication operations including verifying eligibility for the member, identifying/reviewing an applicable formulary for the member to determine any appropriate copayment, coinsurance, and deductible for the prescription drug, and performing a drug utilization review (DUR) for the member. Further, the PBM may provide a response to the pharmacy (for example, the pharmacy system 100) following performance of at least some of the aforementioned operations.

As part of the adjudication, a plan sponsor (or the PBM on behalf of the plan sponsor) ultimately reimburses the pharmacy for filling the prescription drug when the prescription drug was successfully adjudicated. The aforementioned adjudication operations generally occur before the copayment is received and the prescription drug is dispensed. However in some instances, these operations may occur simultaneously, substantially simultaneously, or in a different order. In addition, more or fewer adjudication operations may be performed as at least part of the adjudication process.

The amount of reimbursement paid to the pharmacy by a plan sponsor and/or money paid by the member may be determined at least partially based on types of pharmacy networks in which the pharmacy is included. In some implementations, the amount may also be determined based on other factors. For example, if the member pays the pharmacy for the prescription drug without using the prescription or drug benefit provided by the PBM, the amount of money paid by the member may be higher than when the member uses the prescription or drug benefit. In some implementations, the amount of money received by the pharmacy for dispensing the prescription drug and for the prescription drug itself may be higher than when the member uses the prescription or drug benefit. Some or all of the foregoing operations may be performed by executing instructions stored in the benefit manager device 102 and/or an additional device.

Examples of the network 104 include a Global System for Mobile Communications (GSM) network, a code division multiple access (CDMA) network, 3rd Generation Partnership Project (3GPP), an Internet Protocol (IP) network, a Wireless Application Protocol (WAP) network, or an IEEE 802.11 standards network, as well as various combinations of the above networks. The network 104 may include an optical network. The network 104 may be a local area network or a global communication network, such as the Internet. In some implementations, the network 104 may include a network dedicated to prescription orders: a prescribing network such as the electronic prescribing network operated by Surescripts of Arlington, Virginia.

Moreover, although the system shows a single network 104, multiple networks can be used. The multiple networks may communicate in series and/or parallel with each other to link the devices 102-110.

The pharmacy device 106 may be a device associated with a retail pharmacy location (e.g., an exclusive pharmacy location, a grocery store with a retail pharmacy, or a general sales store with a retail pharmacy) or other type of pharmacy location at which a member attempts to obtain a prescription. The pharmacy may use the pharmacy device 106 to submit the claim to the PBM for adjudication.

Additionally, in some implementations, the pharmacy device 106 may enable information exchange between the pharmacy and the PBM. For example, this may allow the sharing of member information such as drug history that may allow the pharmacy to better service a member (for example, by providing more informed therapy consultation and drug interaction information). In some implementations, the benefit manager device 102 may track prescription drug fulfillment and/or other information for users that are not members, or have not identified themselves as members, at the time (or in conjunction with the time) in which they seek to have a prescription filled at a pharmacy.

The pharmacy device 106 may include a pharmacy fulfillment device 112, an order processing device 114, and a pharmacy management device 116 in communication with each other directly and/or over the network 104. The order processing device 114 may receive information regarding filling prescriptions and may direct an order component to one or more devices of the pharmacy fulfillment device 112 at a pharmacy. The pharmacy fulfillment device 112 may fulfill, dispense, aggregate, and/or pack the order components of the prescription drugs in accordance with one or more prescription orders directed by the order processing device 114.

In general, the order processing device 114 is a device located within or otherwise associated with the pharmacy to enable the pharmacy fulfillment device 112 to fulfill a prescription and dispense prescription drugs. In some implementations, the order processing device 114 may be an external order processing device separate from the pharmacy and in communication with other devices located within the pharmacy.

For example, the external order processing device may communicate with an internal pharmacy order processing device and/or other devices located within the system 100. In some implementations, the external order processing device may have limited functionality (e.g., as operated by a user requesting fulfillment of a prescription drug), while the internal pharmacy order processing device may have greater functionality (e.g., as operated by a pharmacist).

The order processing device 114 may track the prescription order as it is fulfilled by the pharmacy fulfillment device 112. The prescription order may include one or more prescription drugs to be filled by the pharmacy. The order processing device 114 may make pharmacy routing decisions and/or order consolidation decisions for the particular prescription order. The pharmacy routing decisions include what device(s) in the pharmacy are responsible for filling or otherwise handling certain portions of the prescription order. The order consolidation decisions include whether portions of one prescription order or multiple prescription orders should be shipped together for a user or a user family. The order processing device 114 may also track and/or schedule literature or paperwork associated with each prescription order or multiple prescription orders that are being shipped together. In some implementations, the order processing device 114 may operate in combination with the pharmacy management device 116.

The order processing device 114 may include circuitry, a processor, a memory to store data and instructions, and communication functionality. The order processing device 114 is dedicated to performing processes, methods, and/or instructions described in this application. Other types of electronic devices may also be used that are specifically configured to implement the processes, methods, and/or instructions described in further detail below.

In some implementations, at least some functionality of the order processing device 114 may be included in the pharmacy management device 116. The order processing device 114 may be in a client-server relationship with the pharmacy management device 116, in a peer-to-peer relationship with the pharmacy management device 116, or in a different type of relationship with the pharmacy management device 116. The order processing device 114 and/or the pharmacy management device 116 may communicate directly (for example, such as by using a local storage) and/or through the network 104 (such as by using a cloud storage configuration, software as a service, etc.) with the storage device 110.

The storage device 110 may include: non-transitory storage (for example, memory, hard disk, CD-ROM, etc.) in communication with the benefit manager device 102 and/or the pharmacy device 106 directly and/or over the network 104. The non-transitory storage may store order data 118, member data 120, claims data 122, drug data 124, prescription data 126, and/or plan sponsor data 128. Further, the system 100 may include additional devices, which may communicate with each other directly or over the network 104.

The order data 118 may be related to a prescription order. The order data may include type of the prescription drug (for example, drug name and strength) and quantity of the prescription drug. The order data 118 may also include data used for completion of the prescription, such as prescription materials. In general, prescription materials include an electronic copy of information regarding the prescription drug for inclusion with or otherwise in conjunction with the fulfilled prescription. The prescription materials may include electronic information regarding drug interaction warnings, recommended usage, possible side effects, expiration date, date of prescribing, etc. The order data 118 may be used by a high-volume fulfillment center to fulfill a pharmacy order.

In some implementations, the order data 118 includes verification information associated with fulfillment of the prescription in the pharmacy. For example, the order data 118 may include videos and/or images taken of (i) the prescription drug prior to dispensing, during dispensing, and/or after dispensing, (ii) the prescription container (for example, a prescription container and sealing lid, prescription packaging, etc.) used to contain the prescription drug prior to dispensing, during dispensing, and/or after dispensing, (iii) the packaging and/or packaging materials used to ship or otherwise deliver the prescription drug prior to dispensing, during dispensing, and/or after dispensing, and/or (iv) the fulfillment process within the pharmacy. Other types of verification information such as barcode data read from pallets, bins, trays, or carts used to transport prescriptions within the pharmacy may also be stored as order data 118.

The member data 120 includes information regarding the members associated with the PBM. The information stored as member data 120 may include personal information, personal health information, protected health information, etc. Examples of the member data 120 include name, age, date of birth, address (including city, state, and zip code), telephone number, e-mail address, medical history, prescription drug history, etc. In various implementations, the prescription drug history may include a prior authorization claim history—including the total number of prior authorization claims, approved prior authorization claims, and denied prior authorization claims. In various implementations, the prescription drug history may include previously filled claims for the member, including a date of each filled claim, a dosage of each filled claim, the drug type for each filled claim, a prescriber associated with each filled claim, and whether the drug associated with each claim is on a formulary (e.g., a list of covered medication).

In various implementations, the medical history may include whether and/or how well each member adhered to one or more specific therapies. The member data 120 may also include a plan sponsor identifier that identifies the plan sponsor associated with the member and/or a member identifier that identifies the member to the plan sponsor. The member data 120 may include a member identifier that identifies the plan sponsor associated with the user and/or a user identifier that identifies the user to the plan sponsor. In various implementations, the member data 120 may include an eligibility period for each member. For example, the eligibility period may include how long each member is eligible for coverage under the sponsored plan. The member data 120 may also include dispensation preferences such as type of label, type of cap, message preferences, language preferences, etc.

The member data 120 may be accessed by various devices in the pharmacy (for example, the high-volume fulfillment center, etc.) to obtain information used for fulfillment and shipping of prescription orders. In some implementations, an external order processing device operated by or on behalf of a member may have access to at least a portion of the member data 120 for review, verification, or other purposes.

In some implementations, the member data 120 may include information for persons who are users of the pharmacy but are not members in the pharmacy benefit plan being provided by the PBM. For example, these users may obtain drugs directly from the pharmacy, through a private label service offered by the pharmacy, the high-volume fulfillment center, or otherwise. In general, the terms “member” and “user” may be used interchangeably.

The claims data 122 includes information regarding pharmacy claims adjudicated by the PBM under a drug benefit program provided by the PBM for one or more plan sponsors. In general, the claims data 122 includes an identification of the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copayment/coinsurance amount, rebate information, and/or member eligibility, etc. Additional information may be included.

In some implementations, other types of claims beyond prescription drug claims may be stored in the claims data 122. For example, medical claims, dental claims, wellness claims, or other types of health-care-related claims for members may be stored as a portion of the claims data 122.

In some implementations, the claims data 122 includes claims that identify the members with whom the claims are associated. Additionally or alternatively, the claims data 122 may include claims that have been de-identified (that is, associated with a unique identifier but not with a particular, identifiable member). In various implementations, the claims data 122 may include a percentage of prior authorization cases for each prescriber that have been denied, and a percentage of prior authorization cases for each prescriber that have been approved.

The drug data 124 may include drug name (e.g., technical name and/or common name), other names by which the drug is known, active ingredients, an image of the drug (such as in pill form), etc. The drug data 124 may include information associated with a single medication or multiple medications. For example, the drug data 124 may include a numerical identifier for each drug, such as the U.S. Food and Drug Administration's (FDA) National Drug Code (NDC) for each drug.

The prescription data 126 may include information regarding prescriptions that may be issued by prescribers on behalf of users, who may be members of the pharmacy benefit plan—for example, to be filled by a pharmacy. Examples of the prescription data 126 include user names, medication or treatment (such as lab tests), dosing information, etc. The prescriptions may include electronic prescriptions or paper prescriptions that have been scanned. In some implementations, the dosing information reflects a frequency of use (e.g., once a day, twice a day, before each meal, etc.) and a duration of use (e.g., a few days, a week, a few weeks, a month, etc.).

In some implementations, the order data 118 may be linked to associated member data 120, claims data 122, drug data 124, and/or prescription data 126.

The plan sponsor data 128 includes information regarding the plan sponsors of the PBM. Examples of the plan sponsor data 128 include company name, company address, contact name, contact telephone number, contact e-mail address, etc.

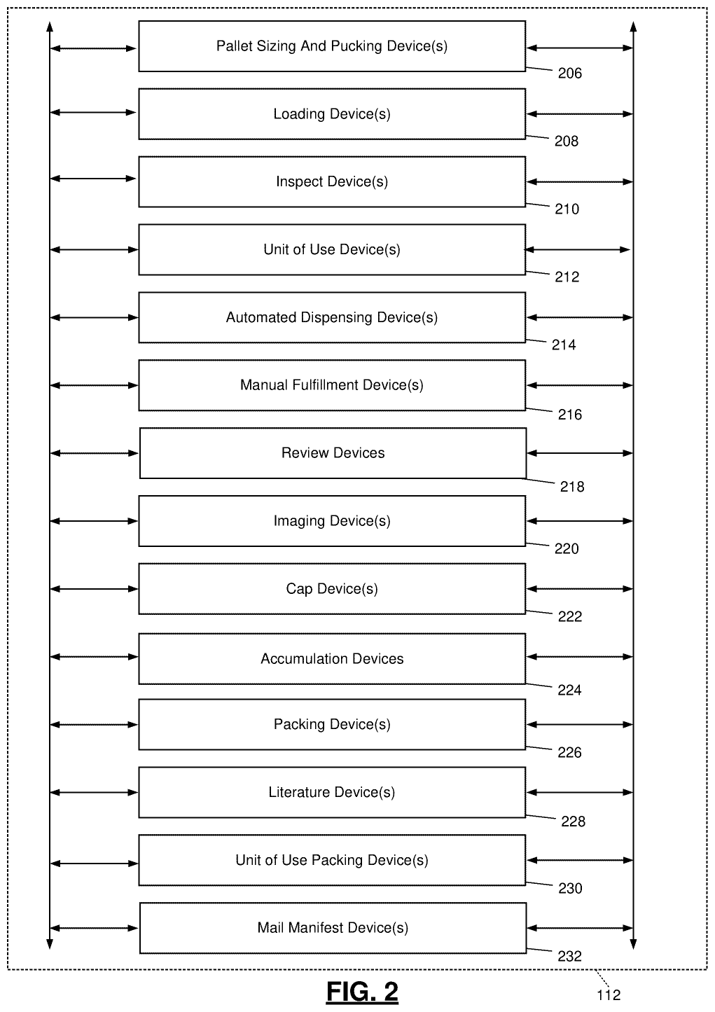

FIG. 2 illustrates the pharmacy fulfillment device 112 according to an example implementation. The pharmacy fulfillment device 112 may be used to process and fulfill prescriptions and prescription orders. After fulfillment, the fulfilled prescriptions are packed for shipping.

The pharmacy fulfillment device 112 may include devices in communication with the benefit manager device 102, the order processing device 114, and/or the storage device 110, directly or over the network 104. Specifically, the pharmacy fulfillment device 112 may include pallet sizing and pucking device(s) 206, loading device(s) 208, inspect device(s) 210, unit of use device(s) 212, automated dispensing device(s) 214, manual fulfillment device(s) 216, review devices 218, imaging device(s) 220, cap device(s) 222, accumulation devices 224, packing device(s) 226, literature device(s) 228, unit of use packing device(s) 230, and mail manifest device(s) 232. Further, the pharmacy fulfillment device 112 may include additional devices, which may communicate with each other directly or over the network 104.

In some implementations, operations performed by one of these devices 206-232 may be performed sequentially, or in parallel with the operations of another device as may be coordinated by the order processing device 114. In some implementations, the order processing device 114 tracks a prescription with the pharmacy based on operations performed by one or more of the devices 206-232.

In some implementations, the pharmacy fulfillment device 112 may transport prescription drug containers, for example, among the devices 206-232 in the high-volume fulfillment center, by use of pallets. The pallet sizing and pucking device 206 may configure pucks in a pallet. A pallet may be a transport structure for a number of prescription containers, and may include a number of cavities. A puck may be placed in one or more than one of the cavities in a pallet by the pallet sizing and pucking device 206. The puck may include a receptacle sized and shaped to receive a prescription container. Such containers may be supported by the pucks during carriage in the pallet. Different pucks may have differently sized and shaped receptacles to accommodate containers of differing sizes, as may be appropriate for different prescriptions.

The arrangement of pucks in a pallet may be determined by the order processing device 114 based on prescriptions that the order processing device 114 decides to launch. The arrangement logic may be implemented directly in the pallet sizing and pucking device 206. Once a prescription is set to be launched, a puck suitable for the appropriate size of container for that prescription may be positioned in a pallet by a robotic arm or pickers. The pallet sizing and pucking device 206 may launch a pallet once pucks have been configured in the pallet.

The loading device 208 may load prescription containers into the pucks on a pallet by a robotic arm, a pick and place mechanism (also referred to as pickers), etc. In various implementations, the loading device 208 has robotic arms or pickers to grasp a prescription container and move it to and from a pallet or a puck. The loading device 208 may also print a label that is appropriate for a container that is to be loaded onto the pallet, and apply the label to the container. The pallet may be located on a conveyor assembly during these operations (e.g., at the high-volume fulfillment center, etc.).

The inspect device 210 may verify that containers in a pallet are correctly labeled and in the correct spot on the pallet. The inspect device 210 may scan the label on one or more containers on the pallet. Labels of containers may be scanned or imaged in full or in part by the inspect device 210. Such imaging may occur after the container has been lifted out of its puck by a robotic arm, picker, etc., or may be otherwise scanned or imaged while retained in the puck. In some implementations, images and/or video captured by the inspect device 210 may be stored in the storage device 110 as order data 118.

The unit of use device 212 may temporarily store, monitor, label, and/or dispense unit of use products. In general, unit of use products are prescription drug products that may be delivered to a user or member without being repackaged at the pharmacy. These products may include pills in a container, pills in a blister pack, inhalers, etc. Prescription drug products dispensed by the unit of use device 212 may be packaged individually or collectively for shipping, or may be shipped in combination with other prescription drugs dispensed by other devices in the high-volume fulfillment center.

At least some of the operations of the devices 206-232 may be directed by the order processing device 114. For example, the manual fulfillment device 216, the review device 218, the automated dispensing device 214, and/or the packing device 226, etc. may receive instructions provided by the order processing device 114.

The automated dispensing device 214 may include one or more devices that dispense prescription drugs or pharmaceuticals into prescription containers in accordance with one or multiple prescription orders. In general, the automated dispensing device 214 may include mechanical and electronic components with, in some implementations, software and/or logic to facilitate pharmaceutical dispensing that would otherwise be performed in a manual fashion by a pharmacist and/or pharmacist technician. For example, the automated dispensing device 214 may include high-volume fillers that fill a number of prescription drug types at a rapid rate and blister pack machines that dispense and pack drugs into a blister pack. Prescription drugs dispensed by the automated dispensing devices 214 may be packaged individually or collectively for shipping, or may be shipped in combination with other prescription drugs dispensed by other devices in the high-volume fulfillment center.

The manual fulfillment device 216 controls how prescriptions are manually fulfilled. For example, the manual fulfillment device 216 may receive or obtain a container and enable fulfillment of the container by a pharmacist or pharmacy technician. In some implementations, the manual fulfillment device 216 provides the filled container to another device in the pharmacy fulfillment devices 112 to be joined with other containers in a prescription order for a user or member.

In general, manual fulfillment may include operations at least partially performed by a pharmacist or a pharmacy technician. For example, a person may retrieve a supply of the prescribed drug, may make an observation, may count out a prescribed quantity of drugs and place them into a prescription container, etc. Some portions of the manual fulfillment process may be automated by use of a machine. For example, counting of capsules, tablets, or pills may be at least partially automated (such as through use of a pill counter). Prescription drugs dispensed by the manual fulfillment device 216 may be packaged individually or collectively for shipping, or may be shipped in combination with other prescription drugs dispensed by other devices in the high-volume fulfillment center.

The review device 218 may process prescription containers to be reviewed by a pharmacist for proper pill count, exception handling, prescription verification, etc. Fulfilled prescriptions may be manually reviewed and/or verified by a pharmacist, as may be required by state or local law. A pharmacist or other licensed pharmacy person who may dispense certain drugs in compliance with local and/or other laws may operate the review device 218 and visually inspect a prescription container that has been filled with a prescription drug. The pharmacist may review, verify, and/or evaluate drug quantity, drug strength, and/or drug interaction concerns, or otherwise perform pharmacist services. The pharmacist may also handle containers which have been flagged as an exception, such as containers with unreadable labels, containers for which the associated prescription order has been canceled, containers with defects, etc. In an example, the manual review can be performed at a manual review station.

The imaging device 220 may image containers once they have been filled with pharmaceuticals. The imaging device 220 may measure a fill height of the pharmaceuticals in the container based on the obtained image to determine if the container is filled to the correct height given the type of pharmaceutical and the number of pills in the prescription. Images of the pills in the container may also be obtained to detect the size of the pills themselves and markings thereon. The images may be transmitted to the order processing device 114 and/or stored in the storage device 110 as part of the order data 118.

The cap device 222 may be used to cap or otherwise seal a prescription container. In some implementations, the cap device 222 may secure a prescription container with a type of cap in accordance with a user preference (e.g., a preference regarding child resistance, etc.), a plan sponsor preference, a prescriber preference, etc. The cap device 222 may also etch a message into the cap, although this process may be performed by a subsequent device in the high-volume fulfillment center.

The accumulation device 224 accumulates various containers of prescription drugs in a prescription order. The accumulation device 224 may accumulate prescription containers from various devices or areas of the pharmacy. For example, the accumulation device 224 may accumulate prescription containers from the unit of use device 212, the automated dispensing device 214, the manual fulfillment device 216, and the review device 218. The accumulation device 224 may be used to group the prescription containers prior to shipment to the member.

The literature device 228 prints, or otherwise generates, literature to include with each prescription drug order. The literature may be printed on multiple sheets of substrates, such as paper, coated paper, printable polymers, or combinations of the above substrates. The literature printed by the literature device 228 may include information required to accompany the prescription drugs included in a prescription order, other information related to prescription drugs in the order, financial information associated with the order (for example, an invoice or an account statement), etc.

In some implementations, the literature device 228 folds or otherwise prepares the literature for inclusion with a prescription drug order (e.g., in a shipping container). In other implementations, the literature device 228 prints the literature and is separate from another device that prepares the printed literature for inclusion with a prescription order.

The packing device 226 packages the prescription order in preparation for shipping the order. The packing device 226 may box, bag, or otherwise package the fulfilled prescription order for delivery. The packing device 226 may further place inserts (e.g., literature or other papers, etc.) into the packaging received from the literature device 228. For example, bulk prescription orders may be shipped in a box, while other prescription orders may be shipped in a bag, which may be a wrap seal bag.

The packing device 226 may label the box or bag with an address and a recipient's name. The label may be printed and affixed to the bag or box, be printed directly onto the bag or box, or otherwise associated with the bag or box. The packing device 226 may sort the box or bag for mailing in an efficient manner (e.g., sort by delivery address, etc.). The packing device 226 may include ice or temperature sensitive elements for prescriptions that are to be kept within a temperature range during shipping (for example, this may be necessary in order to retain efficacy). The ultimate package may then be shipped through postal mail, through a mail order delivery service that ships via ground and/or air (e.g., UPS, FEDEX, or DHL, etc.), through a delivery service, through a locker box at a shipping site (e.g., AMAZON locker or a PO Box, etc.), or otherwise.

The unit of use packing device 230 packages a unit of use prescription order in preparation for shipping the order. The unit of use packing device 230 may include manual scanning of containers to be bagged for shipping to verify each container in the order. In an example implementation, the manual scanning may be performed at a manual scanning station. The pharmacy fulfillment device 112 may also include a mail manifest device 232 to print mailing labels used by the packing device 226 and may print shipping manifests and packing lists.

While the pharmacy fulfillment device 112 in FIG. 2 is shown to include single devices 206-232, multiple devices may be used. When multiple devices are present, the multiple devices may be of the same device type or models, or may be a different device type or model. The types of devices 206-232 shown in FIG. 2 are example devices. In other configurations of the system 100, lesser, additional, or different types of devices may be included.

Moreover, multiple devices may share processing and/or memory resources. The devices 206-232 may be located in the same area or in different locations. For example, the devices 206-232 may be located in a building or set of adjoining buildings. The devices 206-232 may be interconnected (such as by conveyors), networked, and/or otherwise in contact with one another or integrated with one another (e.g., at the high-volume fulfillment center, etc.). In addition, the functionality of a device may be split among a number of discrete devices and/or combined with other devices. These devices may output or generate data that can be fed into the learning systems, prediction systems, artificial intelligence systems, generative artificial intelligence systems, or the like to produce, train or modify the predictive models as described herein.

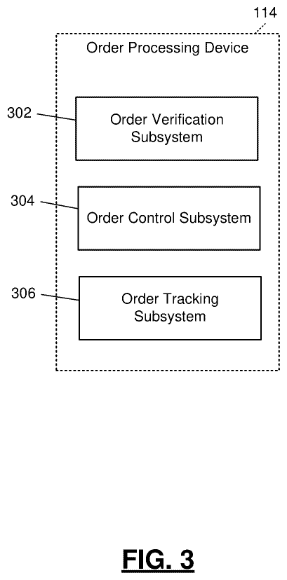

FIG. 3 illustrates the order processing device 114 according to an example implementation. The order processing device 114 may be used by one or more operators to generate prescription orders, make routing decisions, make prescription order consolidation decisions, track literature with the system 100, and/or view order status and other order related information. For example, the prescription order may be comprised of order components.

The order processing device 114 may receive instructions to fulfill an order without operator intervention. An order component may include a prescription drug fulfilled by use of a container through the system 100. The order processing device 114 may include an order verification subsystem 302, an order control subsystem 304, and/or an order tracking subsystem 306. Other subsystems may also be included in the order processing device 114.

The order verification subsystem 302 may communicate with the benefit manager device 102 to verify the eligibility of the member and review the formulary to determine appropriate copayment, coinsurance, and deductible for the prescription drug and/or perform a DUR (drug utilization review). Other communications between the order verification subsystem 302 and the benefit manager device 102 may be performed for a variety of purposes.

The order control subsystem 304 controls various movements of the containers and/or pallets along with various filling functions during their progression through the system 100. In some implementations, the order control subsystem 304 may identify the prescribed drug in one or more than one prescription orders as capable of being fulfilled by the automated dispensing device 214. The order control subsystem 304 may determine which prescriptions are to be launched and may determine that a pallet of automated-fill containers is to be launched.

The order control subsystem 304 may determine that an automated-fill prescription of a specific pharmaceutical is to be launched and may examine a queue of orders awaiting fulfillment for other prescription orders, which will be filled with the same pharmaceutical. The order control subsystem 304 may then launch orders with similar automated-fill pharmaceutical needs together in a pallet to the automated dispensing device 214. As the devices 206-232 may be interconnected by a system of conveyors or other container movement systems, the order control subsystem 304 may control various conveyors: for example, to deliver the pallet from the loading device 208 to the manual fulfillment device 216 from the literature device 228, paperwork as needed to fill the prescription.

The order tracking subsystem 306 may track a prescription order during its progress toward fulfillment. The order tracking subsystem 306 may track, record, and/or update order history, order status, etc. The order tracking subsystem 306 may store data locally (for example, in a memory) or as a portion of the order data 118 stored in the storage device 110.

The subsystems 302, 304, or 306 may output or generate data that can be fed into the learning systems, prediction systems, artificial intelligence systems, generative artificial intelligence systems, or the like to produce, train or modify the predictive models as described herein.

### Developing a Population and Generating Action Hierarchies

An analyst user can create rules via a graphical rules creation user interface. Using a graphical user interface to create predictive data model rules lowers the technical knowledge required from the user creating the rules. In some implemenations, the rules creation user interface represents rules and code segments as blocks. In some implementations, previously created and saved rules can be loaded from a rules module for additional modification. In some implementations, rules are recorded with versioning software in a repository and rules are added to the repository after the rules have met a set of authorization criteria (for example, the system detects that an input has been received corresponding to an approval input). After generating the set of rules, the system generates a population by analyzing a set of data records using the generated set of rules and one or more predictive data models. In some implementations, the rules specify which data models to use in the analysis (either by explicitly selecting a specific model or based on one more factors such as accuracy, speed, applicability, and/or efficacy, etc.). In some implementations, the rules determine where to source data records from. In some implementations, the rules generate a population from the data records based on data in the user records. In some implementations, the rules generate a population based on age, sex, gender, health conditions, prescriptions, home address, proximity to a health service, and/or any other attributes associated with a data record.

After generating the population, the system stratifies the population based on the rules. In some implementations, the rules can include stratification criteria based on a severity of a condition associated with the user records. In some implementations, the condition is indicated by data within the user record. In some implementations, the condition is inferred by the one or more predictive models. The stratification can include any number of classes.

In some implementations, the rules specify an action hierarchy associated with a class. The system determines the classification of a particular data record and associates an action hierarchy with the user record based on the classification. In some implementations, a data record will not be associated with an action hierarchy. For example, a user record associated with a low severity condition may not be assigned an action hierarchy, a second user record with a medium severity condition is assigned a first action hierarchy, and a third user record with a high severity condition is assigned a second hierarchy.

In some implementations, an action hierarchy includes a set of goals, actions, and/or tasks. In some implementations, the goals include improving a condition, preparing for surgery, reducing medication use, increasing exercise or therapy, stopping tobacco use, lowering weight, and/or recovering from surgery, etc. In some implementations, actions include performing exercises, links to media, links to set up or schedule appointments, and/or other health plans. In some implementations, actions are automatically marked as completed once a link has been accessed or an appointment has been made. In some implementations, an action is automatically marked completed based on health data submitted to the user account (such as evidence of a condition change or weight loss).

In some implementations, an action hierarchy includes conditional actions if a set of criteria is met. For example, if a user record is associated with a secondary condition (such as an indication that the user associated with the user record uses tobacco products, has exceeded a recommended weight, has recently had surgery, or is planning to have surgery) the action hierarchy includes actions associated with reducing or mitigating the secondary condition.

Once generated, the action hierarchies are associated with the respective user record and transmitted for the viewing by the users associated with the user accounts. In some implementations, the action hierarchies are viewable from an account profile user interface associated with the user record (such as a web portal user interface). In some implementations, the action hierarchy is displayed with a user assessment. The user assessment includes prompts to verify conditions associated with the user record and verify that the predictive model correctly identified and stratified the user record. After detecting user input corresponding to a response to the user assessment, the input data is transmitted to the execution engine for additional analysis. This allows the system to identify false positives (for example, if the input data indicates that the user record should not be associated with the specified condition) and to update an action hierarchy if a condition changes (for example, user input corresponding to the user assessment indicates that a condition has worsened and therefore the user record must be re-stratified). After the additional analysis, the user record will be re-stratified (or removed from the population entirely) and a new (or no) action hierarchy assigned if necessary.

### Block Diagram

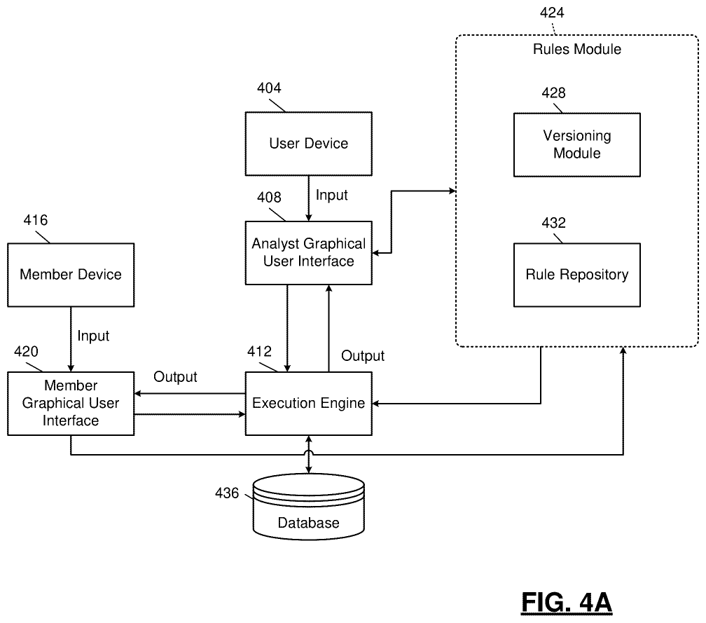

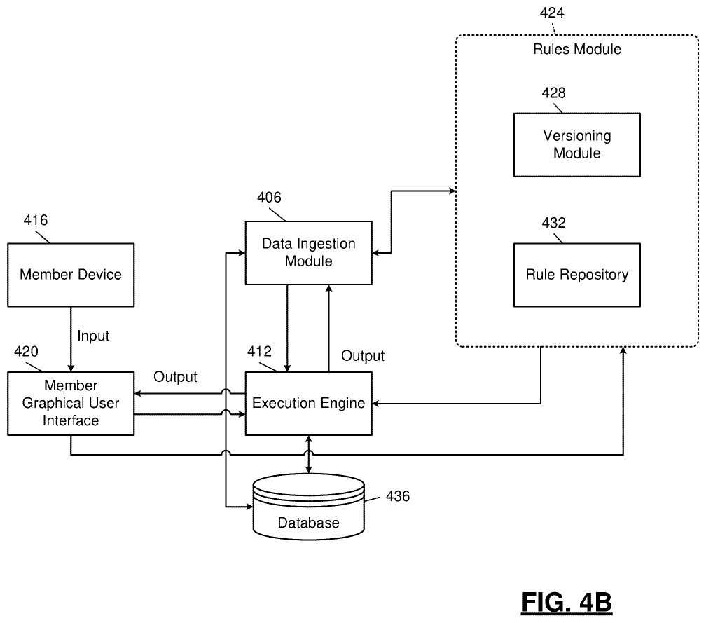

FIG. 4A is a block diagram of an example system that may be used to generate a population and action hierarchies for users. User device 404 (for example, a computer system with a display device, processor, and one or more input devices) displays analyst graphical user interface 408 and detects user input. User device 404 and analyst graphical user interface 408 receive user input corresponding to the creation of rules and a request to generate a population. Analyst graphical user interface 408 transmits rules to rules module 424, which includes versioning module 428 and rule repository 432. Rule repository 432 saves rules created by analyst graphical user interface 408. In some implementations, user device 404 and analyst graphical user interface 408 are used to create rules. In some implementations, user device 404 and analyst graphical user interface 408 are used when creating action hierarchies in real-time. In some implementations, rule repository 432 includes rules previously created by analyst graphical user interface 408 and transmits saved rules to user device 404 for review and/or modification via analyst graphical user interface 408. In some implementations, rules include population generation rules, stratification rules, and/or action hierarchy generation rules. Versioning module 428 manages versions of rules as they are created and modified. Analyst graphical user interface 408 and/or rules module 424 transmits rules to execution engine 412.

Execution engine 412 generates a population based on records and data stored in database 436 (e.g., user records), rules from rules module 424 and/or analyst graphical user interface 408, and one or more predictive models. In some implementations, execution engine 412 stores the predictive models. In some implementations, database 436 stores the predictive models. In some implementations, execution engine 412 stratifies the generated population and creates individualized action hierarchies according to the rules. In some implementations, database 436 includes multiple databases in a plurality of locations. In some implementations, database 436 includes data storage of healthcare providers such as pharmacies, hospitals, insurance providers, and/or primary health care providers. In some implementations, database 436 includes data acquired from external databases.

Execution engine 412 transmits predictions and/or analysis (for example, individualized action hierarchies) to member graphical user interface 420, which is displayed via member device 416. In some implementations, the output of execution engine 412 is transmitted to multiple interfaces on multiple member devices. In some implementations, execution engine 412 transmits output to a server accessible via member device 416 via web portal. In some implementations, member graphical user interface 420 includes displayed user prompts. In some implementations, after member graphical user interface 420 receives user input corresponding to response data to the display user prompts, execution engine 412 receives the response data for analysis. In some implementations, an action hierarchy corresponding to the response data is updated based on the response data.

FIG. 4B is a block diagram of an example system that may be used to generate a population and action hierarchies for users. FIG. 4B includes elements previously discussed with respect to FIG. 4A (such as member device 416, member graphical user interface 420, rules module 424, versioning module 428, rule repository 432, execution engine 412, and database 436). In some implementations, data ingestion module 406 automatically scans for new data and/or data records without user intervention. In some implementations, data ingestion module 406 is used in batch generation of action hierarchies. In some implementations, data ingestion module 406 periodically (for example, daily, weekly, and/or monthly etc.) ingests new data stored in database 436 that requires analysis. In some implementations, data ingestion module 406 instructs execution engine 412 to analyze new data from database 436 using a specific set of rules from rule repository 432. In some implementations, the parameters of data ingestion module 406 are modified by analyst graphical user interface 408. In some implementations, the parameters of data ingestion module 406 are modified via a text editor and/or programming software. In some implementations, the parameters of data ingestion module 406 include how often to ingest new data, which rules to use from rules module 424, and/or which data models to use in execution engine 412, etc.

### Flow Charts

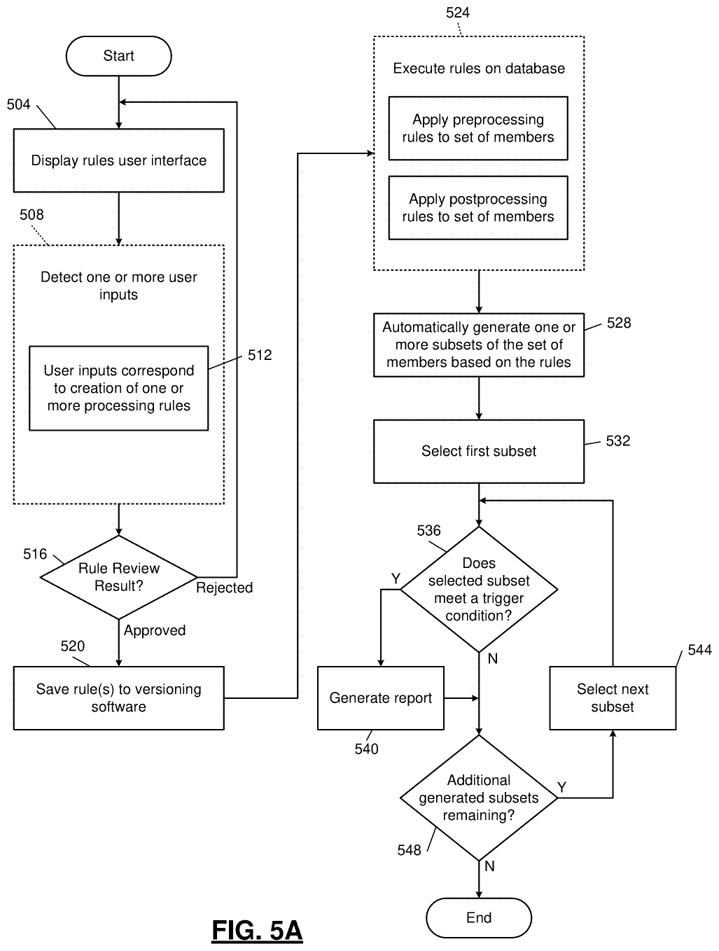

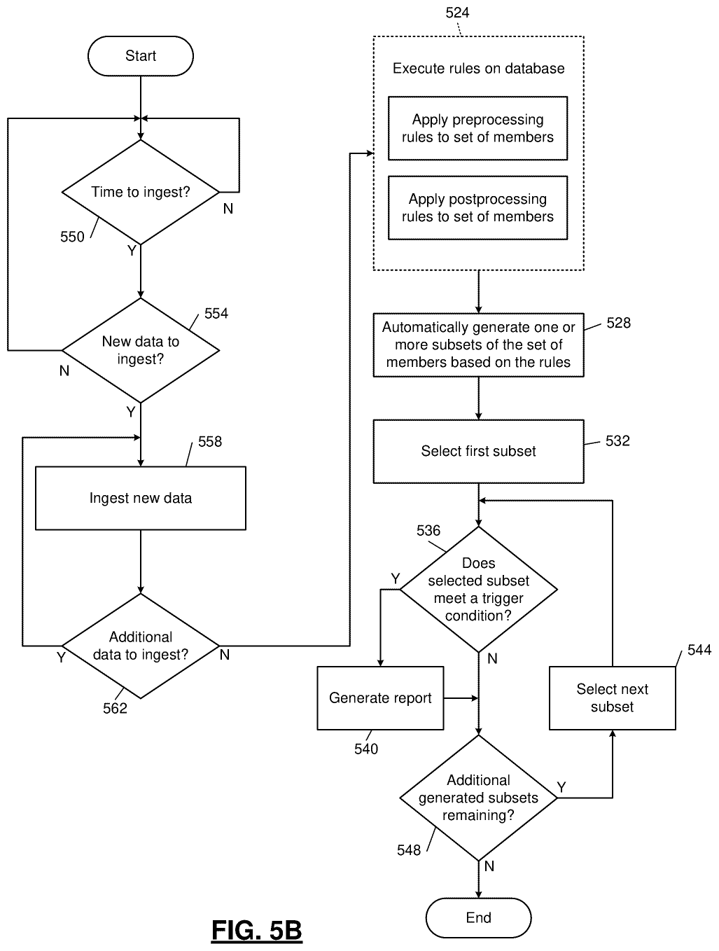

FIG. 5A is a flowchart of an example method of generating a population and individualized action hierarchies for the generated population. The method begins at 504 when control displays a rule user interface for creating and/or modifying rules for the generating a population, stratifying a population, and creating individualized action hierarchies for the stratified population members (for example analyst graphical user interface 408). At 508, control detects one or more user inputs. At 512, control determines that the user inputs correspond to the creation and/or modification of one or more rules for determining the population, stratifying the population, and/or generating an action hierarchy for the population members. At 516, the created and/or modified rules are reviewed (for example for accuracy at versioning module 428). If the rules are rejected, control transfers 504 for additional rules modification/creation. If the rules are approved, control transfers to 520, and the rules are saved (for example to rule repository 432).

Next, control continues to 524 and the rules are executed on data (such as user records). In some implementations, 524 includes executing preprocessing rules for generating a population. In some implementations, 524 includes executing postprocessing rules such as generating statistics on the generated population (for example the size of the population, and/or a predicted accuracy of the population, etc.). After executing the rules on at database at 524, control continues to 528 and generates subsets of the population based on the rules (e.g., stratification of the population). In some implementations, the subsets are based on severity of a condition, the age of user, an overall health score of a user, the accuracy of a prediction, whether an indicated that user is planning or surgery is detected, a prediction of whether a user is going to have surgery, and/or any other method of stratification. At 532, control selects a first subset of the generated population. At 536, control determines whether the selected subset meets a trigger condition (as defined by the created rules). If the selected subset does not meet the trigger condition, control transfers to 548. If the selected subset meets the trigger condition (for example, an age threshold, and/or a condition severity threshold), control transfers to 540 and a report is generated (for example, an action hierarchy) based on the rules and control continues to 548. At 548, control determines if additional subsets have been generated. If false, control ends. If true, control transfers to 544 and selects the next subset and control returns to 536.

FIG. 5B is a flowchart of an example method of generating a population and individualized action hierarchies for the generated population. The method begins at 550. At 550, control determines whether it is time to ingest new data. If the one or more criteria for ingesting new data are not met, control remains at 550. If control determines it is time to ingest new data, control transfers to 554. At 554, control determines if there is new data to ingest. If there is no new data, control transfers to 550. If there is new data, control transfers to 558. At 558, new data is ingested. After 558, control continues to 562 and control determines whether there is additional data to ingest; if so, control returns to 558. If, at 562, there is no additional data to ingest, control transfers to 524. The method continues from 524 to elements 528-548, which may be implemented similarly or identically as described with respect to FIG. 5A.

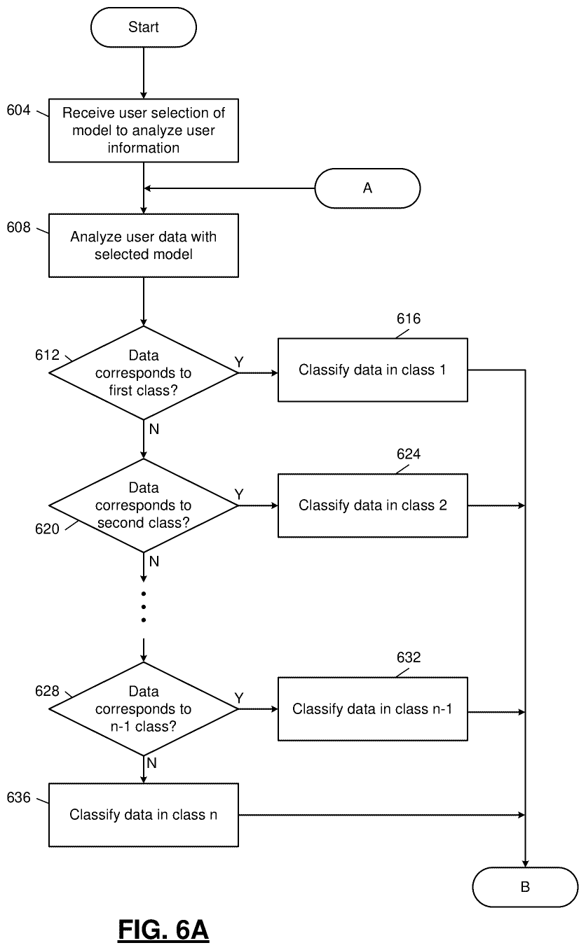

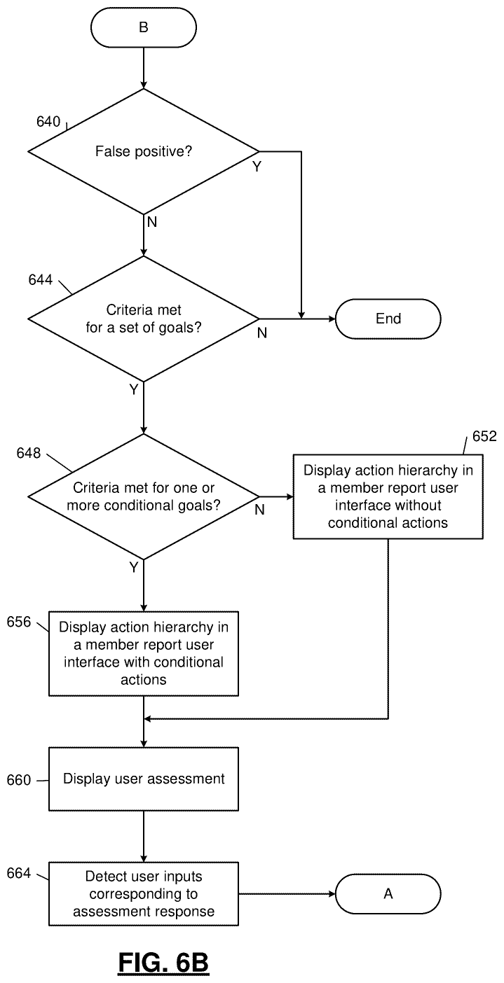

FIGS. 6A-6B are flowchart of an example method of generated individualized action hierarchies for a group of user records and modifying the user records based on user input. At 604, control receives user selection of a data model with which to analyze user information (for example, via analyst graphical user interface 408). At 608, control analyzes the user data with the selected model. In some implementations this includes identifying user records that correspond to a specified condition. In some implementations, the condition is indicated by the user records. In some implementations, the data model infers the condition based on information available in a respective data record. In some implementations, the analysis includes a prediction of behavior of a user associated with a user record. In some implementations, the analysis includes sorting user data (for example by severity of the specified condition).

At 612, control determines if a respective data record corresponds to a first class. If true, control transfers to 616 and the data is classified into class one. If false, control transfers to 620 and determines if the respective data record corresponds to a second class. If true, control transfers to 624 and the data is classified into class two. If false, control continues on, eventually arriving at 628. In some implementations, there are n number of classes, and control performs similar steps and determinations for classes not explicitly shown in FIG. 6A. At 628, control determines if the data corresponds to the n−1 class. If true, control transfers to 632 and classifies the data in the n−1 class. If false, control transfers to 636 and the data is sorted in the n class and control continues to 640.

At FIG. 6B, control continues to 640 to determine if the data is a false positive (for example the data is in an incorrect class and/or should not have been included in the generated population). If true (e.g., a false positive), control ends. If false, control transfers to 644. At 644, control determines if the data has met a set of criteria to be assigned a set of goals (for example, based on the class of the data, the severity of a condition associated with the user record meets one or more thresholds, and/or whether an indication of recent or upcoming surgery is associated with the user record). If the criteria has not been met, control ends. If the criteria has been met, control transfers to 648. In some implementations, the criteria is defined by rules (for example via analyst graphical user interface 408 and/or rules module 424). At 648, control determines if criteria has been by the data for one or more conditional goal. If true, control transfers to 656 and displays an action hierarchy with additional actions in a member report user interface (for example, as part of member graphical user interface 420 on member device 416) and continues 660. If false, control transfers to 652 and displays an action hierarchy without conditional actions and continues to 660. At 660, control displays a user assessment. At 664, control detects user input corresponding to a response to the user assessment and control moves to 608 to analyze the new user data. In some implementations, control performs steps 612-664 multiple times to analyze different data records.

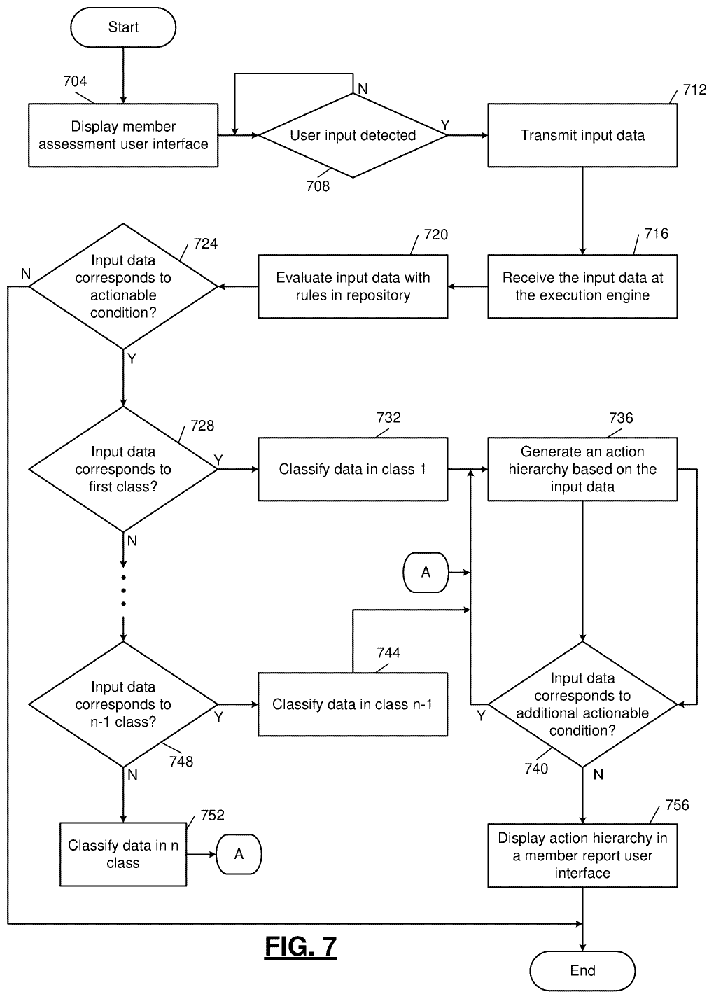

FIG. 7 is flowchart of an example method of generating an action hierarchy for a user record based on user input. At 704 a user assessment is displayed (for example, via member device 416 and member graphical user interface 420) and control continues to 708. At 708, control determines if a user input (for example, user input corresponding to responses to the user assessment) is detected. If false, control remains at 708. If true, control transfers to 712 and control transmits input data to the execution engine (for example, execution engine 412). At 716, the input data is received at the execution engine. At 720, the input data is evaluated by the execution engine following the rules in the rules repository (for example rules repository 432). Control continues to 724 and determines whether the input data corresponds to an actionable condition. If false, control ends. If true, control determines which class the input data corresponds to and classifies the data in steps 728, 732, 748, 744, and 752, which are comparable to steps 612, 616, 628, 632, 620, 624, and 636 as described with respect to FIGS. 6A-6B. After the data has been classified, control continues to 736. At 736, control generates an action hierarchy based on the input data. At 740, control determines if the input data corresponds to an additional actionable condition. If true, control returns to 736. If false, control transfers to 756 and displays an action hierarchy in a member report user interface (such as member graphical user interface 420). After displaying the action hierarchy, control ends.

### Machine Learning

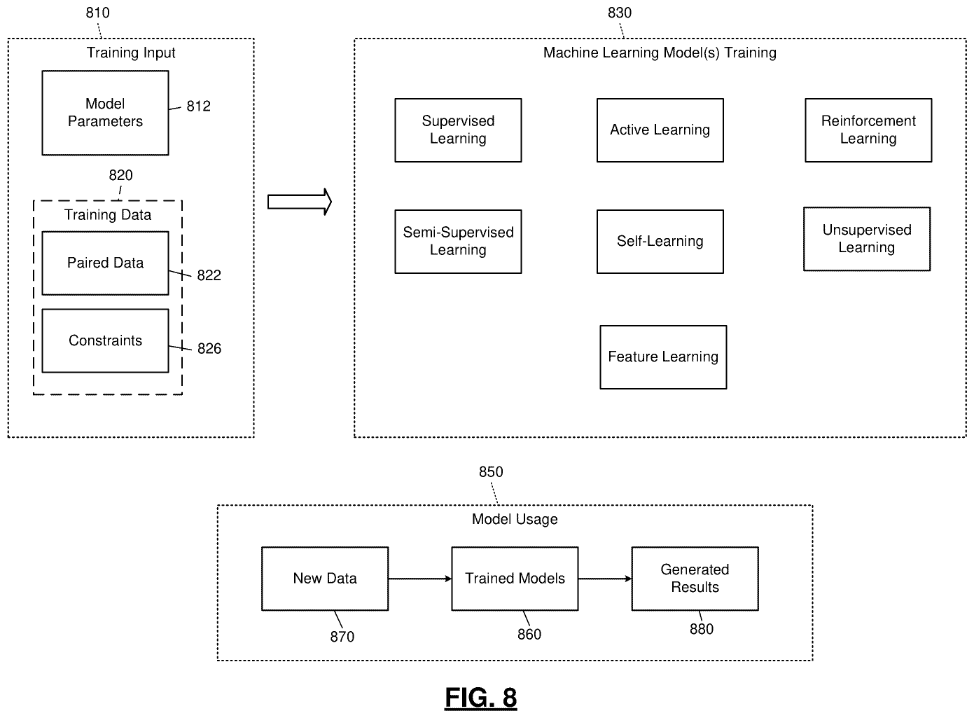

Execution engine 412 uses predictive models to identify individuals to which the user implemented rules apply. For example, FIG. 8 is a block diagram of an example service that may be deployed above. Training input 810 includes model parameters 812 and training data 820, which may include paired training data sets 822 (e.g., input-output training pairs) and constraints 826. Model parameters 812 represents storing and/or providing the parameters or coefficients of corresponding ones of machine learning models. During training, the parameters 812 are adapted based on the input-output training pairs of the training data sets 822. After the parameters 812 are adapted (after training), the parameters are used in 850 by trained models 860 to implement the trained machine learning models on new data 870.

Training data 820 optionally includes constraints 826 which may define the constraints of a given member's information features. The paired training data sets 822 optionally include sets of input-output pairs, such as pairs of a plurality of member preferences and features of entities associated with providers. Some components of training input 810 may be stored separately at a different off-site facility or facilities than other components.

Machine learning model(s) training 830 trains one or more machine learning techniques based on the sets of input-output pairs of paired training data sets 822. For example, the model training 830 may train the machine learning (ML) model parameters 812 by minimizing a loss function based on one or more ground-truth data. The training 830 may include supervised learning, semi-supervised learning, active learning, self-learning, feature learning, reinforcement learning, and unsupervised learning.

The ML models can include any one or combination of classifiers or neural networks, such as an artificial neural network, a convolutional neural network, an adversarial network, a generative adversarial network, a deep feed forward network, a radial basis network, a recurrent neural network, a long/short term memory network, a gated recurrent unit, an auto encoder, a variational autoencoder, a denoising autoencoder, a sparse autoencoder, a Markov chain, a Hopfield network, a Boltzmann machine, a restricted Boltzmann machine, a deep belief network, a deep convolutional network, a deconvolutional network, a deep convolutional inverse graphics network, a liquid state machine, an extreme learning machine, an echo state network, a deep residual network, a Kohonen network, a support vector machine, a neural Turing machine, etc.

Particularly, a first ML model of the ML models can be applied to a training batch of member preferences to estimate or generate a prediction of provider choice for a particular member. In some implementations, a derivative of a loss function is computed based on a comparison of an estimate with ground truth entities, and parameters of the first ML model are updated based on the computed derivative of the loss function. The result of minimizing the loss function for multiple sets of training data trains, adapts, or optimizes the model parameters 812 of the corresponding first ML model. In this way, the first ML model is trained to establish a relationship between member data and member selections.

After the machine learning models are trained, new data 870, including one or more sets of features for members, are received and/or derived from a document being accessed from the storage device 110. The first trained machine learning model may be applied to the new data 870 to generate results 880 (such as a prediction).

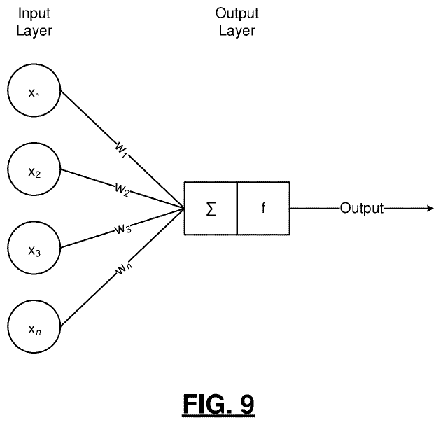

FIG. 9 is a graphical representation of an example neural network with no hidden layers for implementing a machine learning module. In machine learning, a neural network—or an artificial neural network—is a network or circuit of artificial neurons or nodes having at least an input layer and an output layer. In various implementations, neural networks may also have one or more hidden layers. Neural networks may be used in deep learning applications to allow computer systems to solve artificial intelligence problems—such as problems in predictive modeling, pattern recognition, and dynamic control systems.

FIG. 9 shows a neural network without any hidden layers. The neural network of FIG. 9 may also be referred to as a single-layer perceptron. The neural network of FIG. 9 is shown with an input layer including n nodes, labeled x₁, x₂, x₃, and xₙ. While only four nodes are illustrated in FIG. 9, the input layer may have any number of nodes. In various implementations, each node may represent any numerical value. For example, each node may represent a numerical value in a range of between 0 and 1. So, for example, the nodes of the input layer could be expressed in interval notation as: x₁∈[0,1], x₂∈[0,1], x₃ ∈[0,1], and xₙ∈[0,1]. In various implementations, the input variables to a neural network may be expressed as a vector i having n dimensions. In the example of FIG. 9, input vector i may be represented by equation (1) below:

$$i = 〈 {x}_{1} , {x}_{2} , {x}_{3} , {x}_{n} 〉 . \qquad ( 1 )$$

Each of the nodes may be multiplied by a weight—represented by w₁, w₂, w₃, and wₙ in FIG. 9—before being fed into a node in the next layer. In FIG. 9, because there are no hidden layers, the next layer is the output layer. For simplicity of illustration, only a single node is shown in the output layer of FIG. 9. However, the output layer may include any number of nodes.

At the node in the next layer, the inputs of the node are summed. Thus, because the inputs of the node in the output layer of FIG. 9 are the numerical value of each of the nodes of the previous layer multiplied by a weight, the summation Σ may be represented by equation (2) below:

$$\sum = {x}_{1} {w}_{1} + {x}_{2} {w}_{2} + {x}_{3} {w}_{3} + {x}_{n} {w}_{n} . \qquad ( 2 )$$

In various implementations, a bias b may be added to the nodes x of the previous layer after they have been multiplied by a weight w. For example, if biases b are added, then summation Σ may be represented by equation (3) below:

$$\sum = ( {x}_{1} {w}_{1} + {b}_{1} ) + ( {x}_{2} {w}_{2} + {b}_{2} ) + ( {x}_{3} {w}_{3} + {b}_{3} ) + ( {x}_{n} {w}_{n} + {b}_{n} ) \qquad ( 3 )$$

The summation Σ may then be fed into an activation function ƒ. The activation function ƒ may be any mathematical function suitable for calculating an output for the node. Example activation functions ƒ may include linear or non-linear functions, step functions such as the Heaviside step function, derivative or differential functions, monotonic functions, sigmoid or logistic activation functions, rectified linear unit (ReLU) functions, and/or leaky ReLU functions. The output of the function ƒ is then the output of the node. In a neural network with no hidden layers—such as the single-layer perceptron shown in FIG. 9—the output of the nodes in the output layer are the output variables or output vector of the neural network.

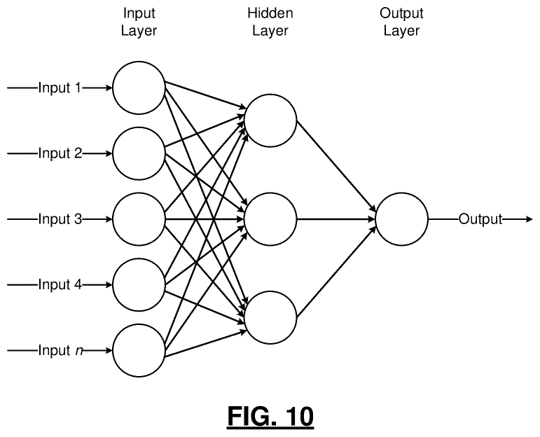

FIG. 10 is a graphical representation of an example neural network with one hidden layer for implementing the machine learning module. As illustrated in FIG. 10, the neural network may one or more intermediate layers—referred to as hidden layers—between the input layer and the output layer. The neural network of FIG. 10 may be referred to as a multilayer perceptron. Each node of a hidden layer may be connected to one or more nodes of the previous layer and receive inputs from the connected nodes of the previous layer—such as the value of the node of the previous layer multiplied by a weight (xₙwₙ) or the value of the node of the previous layer multiplied by a weight with a bias added (xₙwₙ+bₙ). Each node of the hidden layer may then function in a manner analogous to the node of the output layer of FIG. 9 by summing the inputs, feeding the summed inputs into an activation function, and feeding the output of the activation function into one or more nodes of the next layer. Similarly, the nodes of the output layer function in a manner analogous to the node of the output layer of FIG. 9. For example, the nodes of the output layer may receive the outputs of the nodes of the previous layer (multiplied by a weight and/or with a bias added as desired) as inputs, sum the received inputs, feed the summed inputs to an activation function, and output the result of the activation function as an output of the neural network.

In various implementations, the neural network may have any number of hidden layers. In various implementations, each node of a previous layer may be connected to any number of nodes of a next layer. For example, as shown in FIG. 10, each node of the previous layer may be connected to each node of the next layer. Such a neural network may be referred to as a fully-connected neural network. In various implementations, each layer of the neural network may have any number of nodes. In various implementations, a neural network with no hidden layers may function as a linear classifier and be suitable for representing linearly separable decisions or functions. In various implementations, neural networks with one hidden layer may be suitable for performing continuous mapping from one finite space to another. In various implementations, neural networks with two hidden layers may be suitable for approximating any smooth mapping to any level of accuracy.

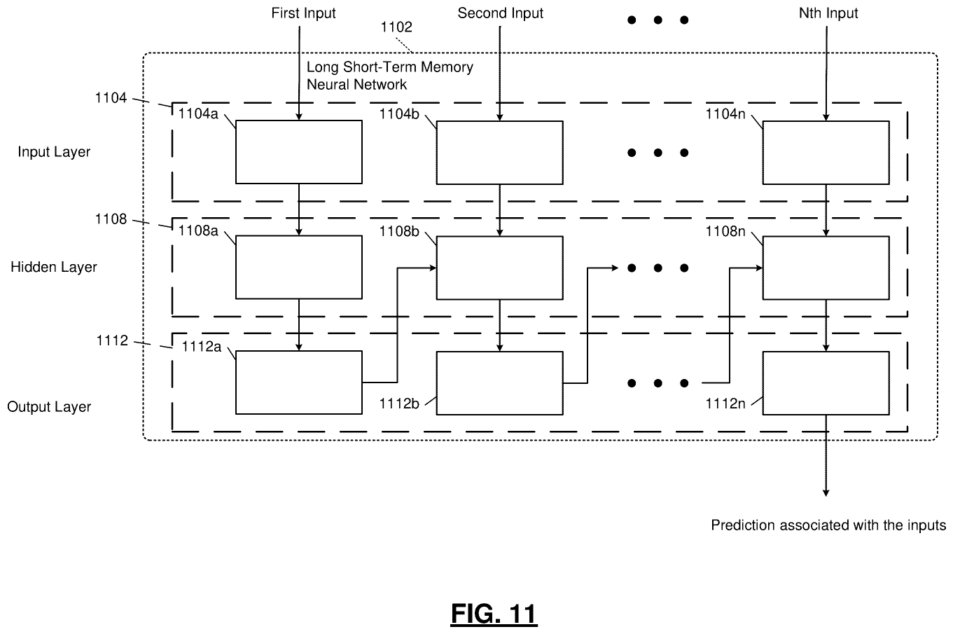

FIG. 11 is a functional block diagram of an example neural network 1102 that can be used to produce a predictive model. In some implementations, the neural network 1102 can be a long short-term memory (LSTM) neural network. In some implementations, the neural network 1102 can be a recurrent neural network (RNN). The example neural network 1102 may be used to implement the machine learning as described herein, and various implementations may use other types of machine learning networks. The neural network 1102 includes an input layer 1104, a hidden layer 1108, and an output layer 1112. The input layer 1104 includes inputs 1104a, 1104b . . . 1104n. The hidden layer 1108 includes neurons 1108a, 1108b . . . 1108n. The output layer 1112 includes outputs 1112a, 1112b . . . 1112n.

Each neuron of the hidden layer 1108 receives an input from the input layer 1104 and outputs a value to the corresponding output in the output layer 1112. For example, the neuron 1108a receives an input from the input 1104a and outputs a value to the output 1112a. Each neuron, other than the neuron 1108a, also receives an output of a previous neuron as an input. For example, the neuron 1108b receives inputs from the input 1104b and the output 1112a. In this way the output of each neuron is fed forward to the next neuron in the hidden layer 1108. The last output 1112n in the output layer 1112 outputs a probability associated with the inputs 1104a-1104n. Although the input layer 1104, the hidden layer 1108, and the output layer 1112 are depicted as each including three elements, each layer may contain any number of elements. Neurons can include one or more adjustable parameters, weights, rules, criteria, or the like.

In various implementations, each layer of the neural network 1102 must include the same number of elements as each of the other layers of the neural network 1102. For example, training features may be processed to create the inputs 1104a-1104n.

The inputs 1104a-1104n can include data features (binary, vectors, factors or the like) stored in the storage device 110. The features can be provided to neurons 1108a-1108n for analysis and connections between the known facts. The neurons 1108a-1108n, upon finding connections, provides the potential connections as outputs to the output layer 1112.

In some examples, a convolutional neural network may be implemented. Similar to neural networks, convolutional neural networks include an input layer, a hidden layer, and an output layer. However, in a convolutional neural network, the output layer includes one fewer output than the number of neurons in the hidden layer and each neuron is connected to each output. Additionally, each input in the input layer is connected to each neuron in the hidden layer. In other words, input 1104a is connected to each of neurons 1108a, 1108b . . . 1108n.

The initial model that is built can be built in a secure environment using health data relating to patients. The initial model can then be refined based on feedback with a computing system that also is in a secure environment. The health data, e.g., the patient name, drug name, dosing data, and other prescription information, is always within a secure computing environment and not communicated out to a public data base and subjected to a third-party artificial intelligence. The secure computing system mitigates the risk of working with protected health data and other types of high-risk data, e.g., personal identifying information, and/or state protected data. In an example, the secure computing system is a mainframe computer with limited connection to external systems. In an example, the computing system is a private cloud environment that provides high-performance, secure, and flexible computing environments enabling the analysis of sensitive datasets restricted by federal privacy laws, proprietary access agreements, or confidentiality requirements. A private cloud environment can provide creation of any combination of network, CPU, RAM, and storage components into resource groups that can be used to build multi-tenant, multi-site infrastructure as a service.

### CONCLUSION

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. In the written description and claims, one or more steps within a method may be executed in a different order (or concurrently) without altering the principles of the present disclosure. Similarly, one or more instructions stored in a non-transitory computer-readable medium may be executed in a different order (or concurrently) without altering the principles of the present disclosure. Unless indicated otherwise, numbering or other labeling of instructions or method steps is done for convenient reference, not to indicate a fixed order.

Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements as well as an indirect relationship where one or more intervening elements are present (either spatially or functionally) between the first and second elements.

The phrase “at least one of A, B, and C” should be construed to mean a logical (A OR B OR C), using a non-exclusive logical OR, and should not be construed to mean “at least one of A, at least one of B, and at least one of C.” The term “set” does not necessarily exclude the empty set—in other words, in some circumstances a “set” may have zero elements. The term “non-empty set” may be used to indicate exclusion of the empty set—in other words, a non-empty set will always have one or more elements. The term “subset” does not necessarily require a proper subset. In other words, a “subset” of a first set may be coextensive with (equal to) the first set. Further, the term “subset” does not necessarily exclude the empty set—in some circumstances a “subset” may have zero elements.

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” can be replaced with the term “controller” or the term “circuit.” In this application, the term “controller” can be replaced with the term “module.”

The term “module” may refer to, be part of, or include processor hardware (shared, dedicated, or group) that executes code and memory hardware (shared, dedicated, or group) that stores code executed by the processor hardware.

The module may include one or more interface circuit(s). In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2020 (also known as the WIFI wireless networking standard) and IEEE Standard 802.3-2018 (also known as the ETHERNET wired networking standard). Examples of a WPAN are IEEE Standard 802.15.4 (including the ZIGBEE standard from the ZigBee Alliance) and, from the Bluetooth Special Interest Group (SIG), the BLUETOOTH wireless networking standard (including Core Specification versions 3.0, 4.0, 4.1, 4.2, 5.0, and 5.1 from the Bluetooth SIG).

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module. For example, the client module may include a native or web application executing on a client device and in network communication with the server module.

The term code, as used above, may include software, firmware, and/or microcode, and may refer to programs, routines, functions, classes, data structures, and/or objects. In an example embodiment, the rules used herein can be a type of code. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

The memory hardware may also store data together with or separate from the code. Shared memory hardware encompasses a single memory device that stores some or all code (e.g., rules and the predictive models) from multiple modules. One example of shared memory hardware may be level 1 cache on or near a microprocessor die, which may store code from multiple modules. Another example of shared memory hardware may be persistent storage, such as a solid-state drive (SSD) or magnetic hard disk drive (HDD), which may store code from multiple modules. Group memory hardware encompasses a memory device that, in combination with other memory devices, stores some or all code from one or more modules. One example of group memory hardware is a storage area network (SAN), which may store code of a particular module across multiple physical devices. Another example of group memory hardware is random access memory of each of a set of servers that, in combination, store code of a particular module.

The term “memory hardware” is a subset of the term computer-readable medium and can include circuitry. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The terms “graphical user interface” or “graphical interface” can refer to processes and data used to visually display on a device, e.g., a computer screen, mobile device screen, monitor, or the like. Such data and processes can interact with the rules, algorithms, predictive models and other functions described herein.

The apparatuses and methods described in this application may be partially or fully implemented by a special purpose computer to execute one or more particular functions embodied in computer programs. Such apparatuses and methods may be described as computerized apparatuses and computerized methods. The functional blocks and flowchart elements described above serve as software specifications, which can be translated into the computer programs by the routine work of a skilled technician or programmer. While the code, rules or predictive models loaded into a machine, e.g., a computer, then that machine or computer is a dedicated machine to the loaded code, rules or predictive model.

Some embodiments described herein are based on rules. The rules can trigger a rule-based system, e.g., a computer system in which domain-specific knowledge is represented in the form of logic sequences and reasoning to perform the functions as described herein. The rules can be codified in a form to be operated on machines. The machines can be a computer as part of a production system, which use if-then rules to derive actions from conditions or a logic programming system, which use conclusion if conditions rules to derive conclusions from conditions. Both kinds of rule-based systems use either forward or backward chaining, or can execute code commands listed sequentially.

The rules can be stored in records and are executable as computer-enabled algorithms including processor-executable instructions that are stored on at least one non-transitory computer-readable medium. The computer algorithms may also include or rely on stored data. The computer algorithms may encompass a basic input/output system (BIOS) that interacts with hardware of the special purpose computer, device drivers that interact with particular devices of the special purpose computer, one or more operating systems, user applications, background services, background applications, and the computing systems described herein.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

1. A computerized method for classifying records across locations, the method comprising:
generating an administrator graphical user interface for presentation to an administrative user at a first user device;
in response to detecting, via the administrator graphical user interface, a set of user inputs from the administrative user:
generating a first set of rules,
transforming the administrator graphical user interface to display a set of user interface elements corresponding to the first set of rules,
transmitting the first set of rules to a first data storage location, and
in response to a determination that the first set of rules have been authorized, storing the first set of rules in the first data storage location;
loading a first set of user records from a set of data storage locations;
generating a second set of user records from the first set of user records using the first set of rules;
determining a classification for a respective record of the second set of user records using the first set of rules, wherein the respective record includes data associated with a second user;
loading a second set of rules from the first data storage location; and
in response to a determination that the classification of the respective record is a first class:
generating a first set of goals based on the second set of rules and the first class, and associating the first set of goals with the respective record,
generating a first set of actions based on the second set of rules and the first class and associating the first set of actions with the respective record, and
transmitting the first set of goals and the first set of actions for access at a second user device.

2. The method of claim 1 further comprising, in response to a determination that the classification of the respective record is a second class:
generating a second set of goals based on the second set of rules and the second class, and associating the second set of goals with the respective record; and
generating a second set of actions based on the second set of rules and the second class and associating the second set of actions with the respective record.

3. The method of claim 2 further comprising:
transmitting the second set of goals and the second set of actions for access at a second user device;
transforming, at the second user device, a second user interface to display at least one of:
the first set of goals and the first set of actions, or
the second set of goals and the second set of actions;
transforming the second user interface to display a first set of user prompts; and
in response to detecting, at the second user device, a second set of user inputs from the second user corresponding to the first set of user prompts:
generating a third set of goals based on the second set of rules and associating the third set of goals with the respective record, and
generating a third set of actions based on the second set of rules and associating the third set of actions with the respective record.

4. The method of claim 3 further comprising:
in response to the second set of user inputs, changing the classification of the respective record to a third class,
wherein the third class is different from the first class and the second class.

5. The method of claim 3 wherein the second set of user inputs corresponds to a set of answers to the first set of user prompts.

6. The method of claim 3 further comprising displaying the first set of user prompts in response to displaying a user account associated with the respective record.

7. The method of claim 6 further compromising:
in response to the determination that the classification of the respective record is the first class, including a first set of questions in the first set of user prompts; and
in response to the determination that the classification of the respective record is the second class, including a second set of questions in the first set of user prompts, wherein at least one question of the second set of questions differs from at least one question of the first set of questions.

8. The method of claim 1 wherein the first data storage location stores data in a non-relational database.

9. The method of claim 1 wherein:
saving data to the first data storage location is performed by a first function; and
loading data from the first data storage location is performed by a second function.

10. The method of claim 1 wherein the set of data storage locations:
is remote from the first data storage location; and
includes a plurality of storage locations.

11. The method of claim 1 further comprising, in response to a determination that the respective record has met one or more criteria of a set of criteria:
generating a fourth set of goals based on the second set of rules and associating the fourth set of goals with the respective record; and
generating a fourth set of actions based on the second set of rules and associating the fourth set of actions with the respective record,
wherein the fourth set of goals and the fourth set of actions are based on a current classification of the respective record.

12. The method of claim 11 wherein the set of criteria includes:
a criterion that is met when the respective record includes an indication of a first health concern;
a criterion that is met when the respective record includes an indication of a second health concern; and
a criterion that is met when the respective record includes an indication of a third health concern.

13. A computer system comprising:
memory hardware configured to store instructions; and
processor hardware configured to execute the instructions, wherein the instructions include:
generating an administrator graphical user interface for presentation to an administrative user at a first user device;
in response to detecting, via the administrator graphical user interface, a set of user inputs from the administrative user:
generating a first set of rules;
transforming the administrator graphical user interface to display a set of user interface elements corresponding to the first set of rules;
transmitting the first set of rules to a first data storage location; and
in response to a determination that the first set of rules have been authorized, storing the first set of rules in the first data storage location;
loading a first set of user records from a set of data storage locations;
generating a second set of user records from the first set of user records using the first set of rules;
determining a classification for a respective record of the second set of user records using the first set of rules, wherein the respective record includes data associated with a second user;
loading a second set of rules from the first data storage location; and
in response to a determination that the classification of the respective record is a first class:
generating a first set of goals based on the second set of rules and the first class, and associating the first set of goals with the respective record;
generating a first set of actions based on the second set of rules and the first class and associating the first set of actions with the respective record; and
transmitting the first set of goals and the first set of actions for access at a second user device.

14. The computer system of claim 13 wherein the instructions include, in response to a determination that the classification of the respective record is a second class:
generating a second set of goals based on the second set of rules and the second class, and associating the second set of goals with the respective record; and
generating a second set of actions based on the second set of rules and the second class and associating the second set of actions with the respective record.

15. The computer system of claim 14 wherein the instructions include:
transmitting the second set of goals and the second set of actions for access at a second user device;
transforming, at the second user device, a second user interface to display at least one of:
the first set of goals and the first set of actions, or
the second set of goals and the second set of actions;
transforming the second user interface to display a first set of user prompts; and
in response to detecting, at the second user device, a second set of user inputs from the second user corresponding to the first set of user prompts:
generating a third set of goals based on the second set of rules and associating the third set of goals with the respective record, and
generating a third set of actions based on the second set of rules and associating the third set of actions with the respective record.

16. The computer system of claim 15 wherein:
the instructions include, in response to the second set of user inputs, changing the classification of the respective record to a third class;
the third class is different from the first class and the second class; and
the second set of user inputs corresponds to a set of answers to the first set of user prompts.

17. A non-transitory computer-readable medium comprising processor-executable instructions, the instructions including:
generating an administrator graphical user interface for presentation to an administrative user at a first user device;
in response to detecting, via the administrator graphical user interface, a set of user inputs from the administrative user:
generating a first set of rules,
transforming the administrator graphical user interface to display a set of user interface elements corresponding to the first set of rules,
transmitting the first set of rules to a first data storage location, and
in response to a determination that the first set of rules have been authorized, storing the first set of rules in the first data storage location;
loading a first set of user records from a set of data storage locations;
generating a second set of user records from the first set of user records using the first set of rules;
determining a classification for a respective record of the second set of user records using the first set of rules, wherein the respective record includes data associated with a second user;
loading a second set of rules from the first data storage location; and
in response to a determination that the classification of the respective record is a first class:
generating a first set of goals based on the second set of rules and the first class, and associating the first set of goals with the respective record,
generating a first set of actions based on the second set of rules and the first class and associating the first set of actions with the respective record, and
transmitting the first set of goals and the first set of actions for access at a second user device.

18. The non-transitory computer-readable medium of claim 17 wherein the instructions include, in response to a determination that the classification of the respective record is a second class:
generating a second set of goals based on the second set of rules and the second class, and associating the second set of goals with the respective record; and
generating a second set of actions based on the second set of rules and the second class and associating the second set of actions with the respective record.

19. The non-transitory computer-readable medium of claim 18 wherein the instructions further include:
transmitting the second set of goals and the second set of actions for access at a second user device;
transforming, at the second user device, a second user interface to display:
the first set of goals and the first set of actions, or
the second set of goals and the second set of actions;
transforming the second user interface to display a first set of user prompts; and
in response to detecting, at the second user device, a second set of user inputs from the second user corresponding to the first set of user prompts:
generating a third set of goals based on the second set of rules and associating the third set of goals with the respective record, and
generating a third set of actions based on the second set of rules and associating the third set of actions with the respective record.

20. The non-transitory computer-readable medium of claim 19 wherein:
the instructions include, in response to the second set of user inputs, changing the classification of the respective record to a third class;
the third class is different from the first class and the second class; and
the second set of user inputs corresponds to a set of answers to the first set of user prompts.
